(19) United States
(12) Patent Application Publication
(10) Pub. No.: US 2011/0178305 A1
(43) Pub. Date: Jul. 21, 2011

(54) PROCESS FOR THE PREPARATION OF 3,4-EPOXY-2-AMINO-1-SUBSTITUTED BUTANE DERIVATIVES AND INTERMEDIATE COMPOUNDS THEREOF

(75) Inventors: Rakesh Singh, Jaunpur (IN); Prasad Yeragorla, Khammam (IN); Mahavir Singh Khanna, New Delhi (IN); Mohan Prasad, Gurgaon (IN)

(73) Assignee: RANBAXY LABORATORIES LIMITED, New Delhi, Delhi (IN)

(21) Appl. No.: 12/991,635

(22) PCT Filed: May 5, 2009
(86) PCT No.: PCT/IB2009/051843

§ 371 Date: Apr. 1, 2011

(30) Foreign Application Priority Data
May 8, 2008 (IN) 1147/DEL/2008

(51) Int. Cl. C07D 213/55; C07C 231/02; C07D 301/02; C07C 261/00; C07C 237/00

(52) U.S. Cl. 546/335; 564/138; 549/519; 560/29; 564/194

(57) ABSTRACT

The present invention relates to a process for the preparation of threo-3,4-epoxy-2-amino-1-substituted butane derivatives represented by general Formula I which comprises reacting compound of Formula III or salt thereof with an active ester of acid of Formula IV and treating the product thereof with base. The carbon atom bonded to the radical R³ in Formula I and IV is in the (R)-, (S)- or (R,S)-configuration. The compounds of Formula I and III, particularly in their (2S,3R) configuration are useful intermediates for the preparation of atazanavir bisulfate.

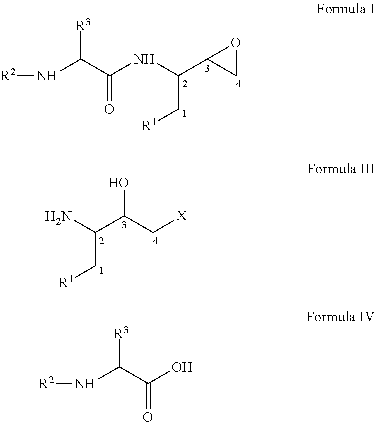

### FIELD OF THE INVENTION

The present invention relates to production method of threo-3,4-epoxy-2-amino-1-substituted butane derivatives represented by general Formula I:

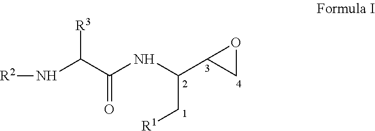

wherein

R¹ is phenyl,

R² is hydrogen or amino protecting groups,

R³ is secondary or tertiary lower alkyl and configurations at 2 and 3 positions are either (2S,3R) or (2R,3S).

The carbon atom bonded to the radical R³ in Formula I may be in (R)-, (S)- or (R,S)-configuration.

The compounds of Formula I are useful intermediates for the production of various HIV protease compounds. Particularly, (2S,3R)-3,4-epoxy-2-amino-1-substituted butane derivatives represented by general Formula Ia are useful pharmaceutical intermediates of atazanavir—an inhibitor of retroviral aspartate protease.

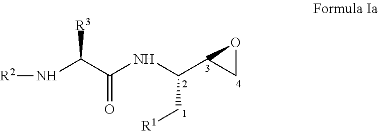

The R¹, R² and R³ in Formula Ia are same as described hereinabove for compound of Formula I.

### BACKGROUND OF THE INVENTION

Atazanavir and its bisulfate salt (1:1) are disclosed in U.S. Pat. Nos. 5,849,911 and 6,087,383 respectively. Atazanavir bisulfate is chemically known as (3S,8S,9S,12S)-3,12-bis(1,1-dimethylethyl)-8-hydroxy-4,11-dioxo-9-(phenylmethyl)-6-[[4-(2-pyridinyl)phenyl]methyl]-2,5,6,10,13-pentaazatetradecanedioic acid dimethyl ester, sulfate (1:1) and represented by following chemical structure—

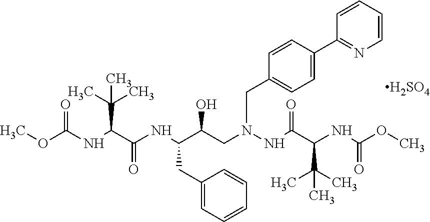

Atazanavir bisulfate is inhibitor of retroviral aspartate protease and also known to have high degree of inhibitory activity against the HIV virus.

Several methods for preparing various 3,4-epoxy-2-amino-1-substituted butane derivatives are published. U.S. Pat. Nos. 5,849,911; 6,300,519 and 6,110,946 describe preparation of 3,4-epoxy-2-amino-1-substituted butane derivatives starting from 2-amino-3-substituted propanoic acid derivative. The acid is reduced to corresponding aldehyde which on further treatment with ylide compound (e.g. sulfur ylide compound) produces the desired 3,4-epoxy-2-amino-1-substituted butane derivative.

U.S. Pat. No. 5,847,169 describes process for preparing 3,4-epoxy-2-amino-1-substituted butane derivatives comprising the steps of activating an aminodiol, acylating the aminodiol and reacting the acylated aminodiol with a base to form an epoxy compound. The methods disclosed in U.S. Pat. No. 6,127,556 for the preparation of epoxy compounds or 3,4-epoxy-2-amino-1-substituted butane derivatives make use of halomethyl organometallic reagent and aminoaldehyde compound whereas U.S. Pat. No. 6,278,002 describes the preparation of similar type of compounds by making use of quaternary ammonium salt or carboxylic acid metal salt. U.S. Pat. No. 6,693,205 makes use of Mitsunobu reaction during preparation of 3,4-epoxy-2-amino-1-substituted butane derivatives.

Several other publications, for example, U.S. Pat. Nos. 5,693,847; 6,344,572; 6,764,545; 6,765,100; 6,867,311; 6,605,732 and 7,122,696 mention preparation of various 3,4-epoxy-2-amino-1-substituted butane derivatives and are incorporated herein by reference.

Most of the prior-art processes for the preparation of 3,4-epoxy-2-amino-1-substituted butane derivatives suffer from one or more disadvantages such as use of expensive and inaccessible raw materials, commercially impractical and hazardous reaction conditions, time consuming multi-step reaction sequences employing unstable and/or dangerous intermediates and production of isomeric mixtures resulting in low yields of pure substance due to lengthy separation procedures that are impractical for larger scale production.

### SUMMARY OF THE INVENTION

The present invention overcomes the disadvantages associated with the prior art by providing a process for the preparation of compounds of the Formula I using reagents and conditions which are convenient to operate on commercial scale and operationally safe.

The threo-4-halo-3-hydroxy-2-amino-1-substituted butane derivative represented by general Formula II serves as substrate in the preparation method of 3,4-epoxy-2-amino-1-substituted butane derivatives (Formula-I) of present invention.

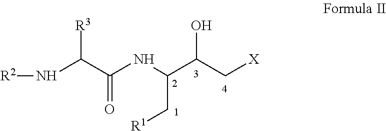

The R¹, R², R³ in Formula-II are same as mentioned hereinabove for the compounds of Formula I and X represents halogen atom, such as a chlorine, bromine, fluorine or iodine.

The carbon atom bonded to the radical R³ in Formula II, may be in the (R)-, (S)- or (R,S)-configuration.

The configurations at positions 2 and 3 in the above Formula II are either (2S,3R) or (2R,3S). The preferred configuration is (2S,3R) as represented by Formula IIa

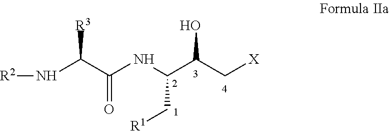

To prepare threo-3,4-epoxy-2-amino-1-substituted butane derivatives having (2S,3R) configuration (Formula-Ia), the above threo-4-halo-3-hydroxy-2-amino-1-substituted butane derivative should have the (2S,3R) configuration (Formula-IIa).

Similarly, for obtaining the (2R,3S) product, the substrate should be of (2R,3S) configuration.

Thus, the present invention provides efficient process for the preparation of compounds of the Formulae I and II.

### DETAILED DESCRIPTION OF THE INVENTION

The term “about” as used herein (unless specified), when used along values assigned to certain measurements and parameters means a variation of 10% from such values, or in case of a range of values, means a 10% variation from both the lower and upper limits of such ranges.

The term ‘lower alkyl’ is meant for ‘C₁-C₄ alkyl’. Preferably, the ‘lower alkyl’ is selected from the group comprising of tert-butyl, sec-butyl, isobutyl, n-butyl, isopropyl, n-propyl, ethyl and methyl.

The term ‘amino protecting groups’ can be any protecting group known to a person skilled in the art. Some non-limiting examples are lower alkoxycarbonyl (such as tert-butoxycarbonyl, methoxycarbonyl etc.), aryl-lower alkoxycarbonyl (such as benzyloxycarbonyl) or acyl protecting group (such as CH₃CO, trifluoroacetyl).

The term ‘Atazanavir bisulfate’ as employed herein refers to Atazanavir bisulfate as well as Atazanavir sulfate.

A first aspect of the present invention provides process for the preparation of 4-halo-3-hydroxy-2-amino-1-substituted butane derivative represented by Formula II

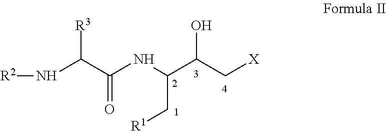

which comprises reacting compound of Formula III or salt thereof

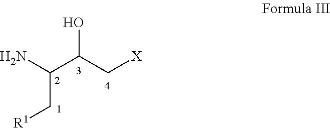

with an active ester of acid of Formula IV

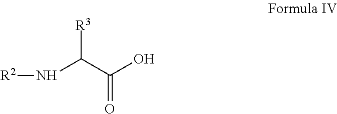

wherein

R¹ is phenyl,

R² is hydrogen or amino protecting groups,

R³ is secondary or tertiary lower alkyl and

X is chlorine, bromine, fluorine or iodine.

In an embodiment of this aspect, the compounds of Formula II and III are either in (2S,3R) or (2R,3S) configuration.

In another embodiment of this aspect, the carbon atom bonded to the radical R³ in Formula II and IV can be in (R)-, (S)- or (R,S)-configuration.

In another embodiment of this aspect, the reaction can be performed in presence of base and organic solvent.

The ‘base’ as used herein can be selected from the group comprising of alkali metal hydroxide, alkaline earth metal hydroxide, alkali metal carbonate, alkaline earth metal carbonate, alkali metal phosphate and alkaline earth metal phosphate. The base can be an organic base. Some non-limiting examples of base are NaOH, KOH, Mg(OH)₂, K₂HPO₄, MgCO₃, Na₂CO₃, K₂CO₃, triethylamine, diisopropylethylamine and/or N-methyl morpholine.

The ‘organic solvent’ as used herein can be selected from the group comprising methylene chloride, ethyl acetate, butyl acetate, dichloroethane, tetrahydrofuran, acetonitrile and N,N-dimethylformamide or mixture(s) thereof.

The acid of Formula IV can be converted into its active ester by reaction of the acid with coupling agent selected from the group comprising of O-(1,2-dihydro-2-oxo-1-pyridyl)-N,N,N¹,N¹-tetramethyluronium-tetrafluoro-borate (TPTU), 1-hydroxybenzotriazole (HOBT) and N-ethyl-N′-dimethylaminopropyl carbodiimide (EDC).

In another embodiment of this aspect, the active ester of an acid of Formula IV can be represented by following compound of Formula V

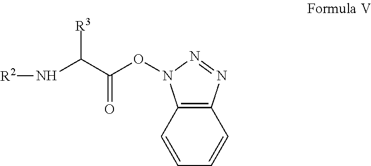

In another embodiment of this aspect, the reaction of compound of Formula III with active ester of acid of Formula IV can be performed at temperature selected from about 5° C. to about 40° C.

In another embodiment of this aspect, the compound of Formula III in its hydrochloride salt form can be reacted with active ester of compound of Formula IV.

Accordingly, the compound of Formula III in its salt form is reacted with active ester of acid of Formula IV in presence of base and organic solvent at temperature selected from about 5° C. to about 40° C. The reaction is performed at pH range of 5 to 7. The so produced compound of Formula II is then isolated from the reaction mixture.

In another embodiment of this aspect, the obtained 4-halo-3-hydroxy-2-amino-1-substituted butane derivative of Formula II has (2S,3R) configuration and can be represented by Formula-IIa.

The compounds of Formula II and IIa can be further used as intermediates in the preparation of Atazanavir or salt thereof.

A second aspect of the present invention provides process for preparation of threo-3,4-epoxy-2-amino-1-substituted butane derivative represented by Formula I:

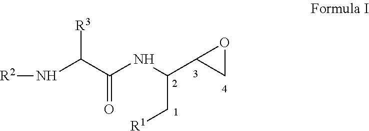

which comprises
a) reacting compound of Formula III or salt thereof

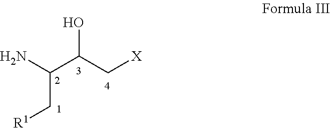

with active ester of acid of Formula IV

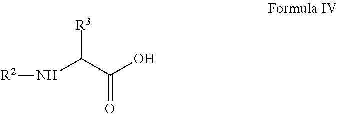

to produce 4-halo-3-hydroxy-2-amino-1-substituted butane derivative of Formula II

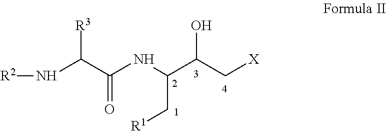

b) treating compound of Formula II produced in step a) with base
c) isolating threo-3,4-epoxy-2-amino-1-substituted butane derivative (I) from the reaction mixture of step b)

wherein

R¹ is phenyl,

R² is hydrogen or amino protecting groups,

R³ is secondary or tertiary lower alkyl and

X is chlorine, bromine, fluorine or iodine.

In an embodiment of this aspect, the compounds of Formula I, II and III can be in (2S,3R) or (2R,3S) configuration.

In another embodiment of this aspect, the carbon atom bonded to the radical R³ in Formula I, II and IV can be in (R)-, (S)- or (R,S)-configuration.

In another embodiment of this aspect, the step a) can be performed in presence of base and organic solvent.

The ‘base’ as used herein in steps a) and b) can be selected from the group comprising alkali metal hydroxide, alkaline earth metal hydroxide, alkali metal carbonate, alkaline earth metal carbonate, alkali metal phosphate and alkaline earth metal phosphate. The base can be an organic base. Some non-limiting examples of base are NaOH, KOH, Mg(OH)₂, K₂HPO₄, MgCO₃, Na₂CO₃, K₂CO₃, triethylamine, diisopropylethylamine and/or N-methyl morpholine.

The ‘organic solvent’ as used herein in step a) can be selected from the group comprising methylene chloride, ethyl acetate, butyl acetate, dichloroethane, tetrahydrofuran, acetonitrile and/or N,N-dimethylformamide.

The acid of Formula IV can be converted into its active ester by the reaction of the acid with coupling agent selected from the group comprising of O-(1,2-dihydro-2-oxo-1-pyridyl)-N,N,N¹,N¹-tetramethyluronium-tetrafluoro-borate (TPTU), 1-hydroxybenzotriazole (HOBT) and N-ethyl-N′-dimethylaminopropyl carbodiimide (EDC).

In another embodiment of this aspect, the active ester of an acid of Formula IV can be represented by following compound of Formula V

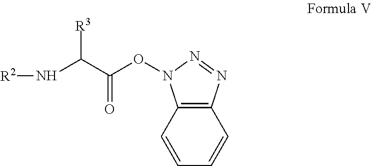

In another embodiment of this aspect, the step a) reaction can be performed at temperature selected from about 5° C. to about 40° C.

In another embodiment of this aspect, the compound of Formula III can be reacted with compound of Formula IV in the form of its hydrochloride salt.

In another embodiment of this aspect, 4-halo-3-hydroxy-2-amino-1-substituted butane derivative compound of Formula II obtained in step a) has (2S,3R) configuration.

In another embodiment of this aspect, the step b) can be performed in presence of polar organic solvent. The polar organic solvent with or without water can be used.

The ‘polar organic solvent’ is not particularly restricted but includes, among others, aprotic polar organic solvents such as acetone, methyl ethyl ketone, tetrahydrofuran, 1,4-dioxane, 1,3-dioxolane, 1,2-dimethoxyethane, diethylene glycol dimethyl ether, trimethylene glycol dimethyl ether, tetraethylene glycol dimethyl ether, polyethylene glycol dimethyl ether, 1,2-diethoxyethane, diethylene glycol diethyl ether, triethylene glycol diethyl ether, tetraethylene glycol diethyl ether, polyethylene glycol diethyl ether, acetonitrile, dimethyl formamide and dimethyl sulfoxide or mixture(s) thereof; protic polar organic solvents such as alcohols, for example methanol, ethanol, n-propanol, isopropanol, n-butanol, sec-butanol, isobutanol and tert-butanol or mixture(s) thereof.

In another embodiment of this aspect, the step b) can be performed at temperature selected from 0° C. to 10° C.

In another embodiment of this aspect, compound of Formula I can be isolated from the reaction mixture by extracting it with ether solvent (e.g. diethyl ether).

Accordingly, the compound of Formula III in its salt form is reacted with an active ester of acid of Formula IV in presence of base and organic solvent at a temperature from about 5° C. to about 40° C. The reaction is performed at pH range of 5 to 7. The so produced compound of Formula II is then isolated from the reaction mixture.

The compound of Formula II is treated with base in polar organic solvent and water to produce the desired threo-3,4-epoxy-2-amino-1-substituted butane derivative of Formula I.

The so produced compound of Formula I is then isolated from the reaction mixture after neutralizing the reaction mixture by adding sodium dihydrogen orthophosphate solution into it. Ether (e.g. diethyl ether) can be added to the reaction mixture to extract the compound of Formula I from the organic layer. The extracted compound of Formula I can be purified using hydrocarbon solvent (e.g. hexane).

The compound of Formula I can be further used as intermediates for the preparation of Atazanavir or salt thereof.

A third aspect of the present invention provides process for the preparation of methyl [(2S)-1-{[(2S,3R)-4-chloro-3-hydroxy-1-phenylbutan-2-yl]amino}-3,3-dimethyl-1-oxobutan-2-yl]carbamate represented by Formula VI

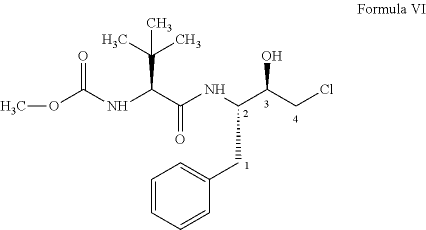

which comprises reacting (2S,3R)-2-amino-4-chloro-1-phenylbutan-3-ol represented by Formula VII or salt thereof

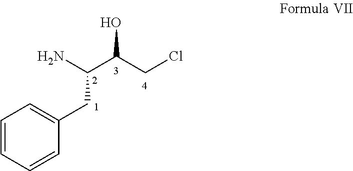

with an active ester of (2S)-2-[(methoxycarbonyl)amino]-3,3-dimethylbutanoic acid represented by Formula VIII.

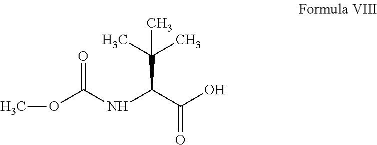

In an embodiment of this aspect, the reaction of compound of Formula VII or salt thereof with an active ester of acid of Formula VIII can be performed in presence of base and organic solvent.

The base and organic solvent used herein in this aspect are the same as mentioned in first aspect of the present invention. The base and organic solvent used in first aspect for the preparation of compound of Formula II comprising reaction of compound of Formula III or salt thereof with active ester of acid of Formula IV can also be employed herein in this aspect of the invention but for the preparation of compound of Formula VI comprising reaction of compound of Formula VII or salt thereof with active ester of acid of Formula VIII.

The acid of Formula VIII can be converted into its active ester by the reaction of the acid with coupling agent selected from the group comprising of O-(1,2-dihydro-2-oxo-1-pyridyl)-N,N,N¹,N¹-tetramethyluronium-tetrafluoro-borate (TPTU), 1-hydroxybenzotriazole (HOBT) and N-ethyl-N′-dimethylaminopropyl carbodiimide (EDC).

In another embodiment of this aspect, the active ester of an acid of Formula VIII can be represented by following Formula IX

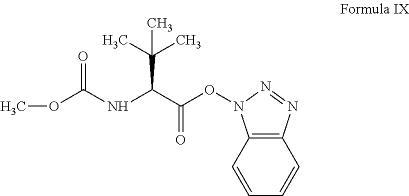

In another embodiment of this aspect, the reaction of compound of Formula VII or salt thereof with active ester of acid of Formula VIII can be performed at temperature selected from about 5° C. to about 40° C.

In another embodiment of this aspect, the compound of Formula VII in its hydrochloride salt form can be reacted with active ester of compound of Formula VIII.

Accordingly, the compound of Formula VII in its salt form is reacted with an active ester of acid of Formula VIII in presence of base and organic solvent at temperature selected from about 5° C. to about 40° C. The reaction is performed at pH range of 5 to 7. The so produced compound of Formula VI is then isolated from the reaction mixture.

The compound of Formula VI can be used as an intermediate in the preparation of Atazanavir or salt thereof.

A fourth aspect of the present invention provides process for the preparation of methyl [(2S)-3,3-dimethyl-1-({(1S)-1-[(2R)-oxiran-2-yl]-2-phenylethyl}amino)-1-oxobutan-2-yl]carbamate represented by Formula X:

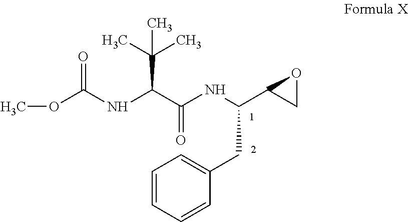

which comprises
a) reacting (2S,3R)-2-amino-4-chloro-1-phenylbutan-3-ol represented by Formula VII or salt thereof

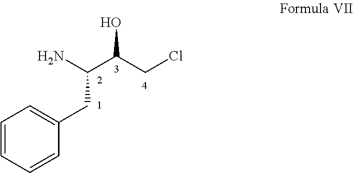

with an active ester of (2S)-2-[(methoxycarbonyl)amino]-3,3-dimethylbutanoic acid represented by Formula VIII

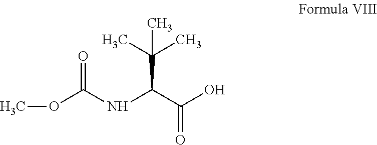

to produce methyl [(2S)-1-{[(2S,3R)-4-chloro-3-hydroxy-1-phenylbutan-2-yl]amino}-3,3-dimethyl-1-oxobutan-2-yl]-carbamate represented by Formula VI

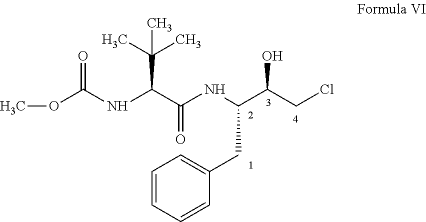

b) treating compound of Formula VI produced in step a) with base
c) isolating methyl [(2S)-3,3-dimethyl-1-({(1S)-1-[(2R)-oxiran-2-yl]-2-phenylethyl}amino)-1-oxobutan-2-yl]-carbamate represented by Formula X from the reaction mixture of step b).

In another embodiment of this aspect, the step a) can be performed in presence of base and organic solvent.

The ‘base’ as used herein in steps a) and b) can be selected from the group comprising alkali metal hydroxide, alkaline earth metal hydroxide, alkali metal carbonate, alkaline earth metal carbonate, alkali metal phosphate and alkaline earth metal phosphate. The base can be an organic base. Some non-limiting examples of base are NaOH, KOH, Mg(OH)₂, K₂HPO₄, MgCO₃, Na₂CO₃, K₂CO₃, triethylamine, diisopropylethylamine and/or N-methyl morpholine.

The ‘organic solvent’ as used herein in step a) can be selected from the group comprising methylene chloride, ethyl acetate, butyl acetate, dichloroethane, tetrahydrofuran, acetonitrile and/or N,N-dimethylformamide.

The acid of Formula VIII can be converted into its active ester by the reaction of the acid with coupling agent selected from the group comprising of O-(1,2-dihydro-2-oxo-1-pyridyl)-N,N,N¹,N¹-tetramethyluronium-tetrafluoro-borate (TPTU), 1-hydroxybenzotriazole (HOBT) and N-ethyl-N′-dimethylaminopropyl carbodiimide (EDC).

In another embodiment of this aspect, the active ester of an acid of Formula VIII can be represented by following Formula IX

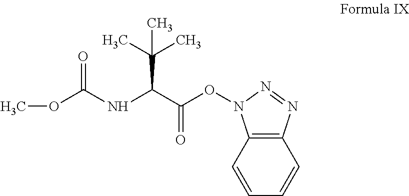

In another embodiment of this aspect, the step a) reaction can be performed at temperature selected from about 5° C. to about 40° C.

In another embodiment of this aspect, the compound of Formula VII in its hydrochloride salt form can be reacted with active ester of compound of Formula VIII.

In another embodiment of this aspect, the step b) can be performed in presence of polar organic solvent. The polar organic solvent with or without water can be used.

The ‘polar organic solvent’ is not particularly restricted but includes, among others, aprotic polar organic solvents such as acetone, methyl ethyl ketone, tetrahydrofuran, 1,4-dioxane, 1,3-dioxolane, 1,2-dimethoxyethane, diethylene glycol dimethyl ether, trimethylene glycol dimethyl ether, tetraethylene glycol dimethyl ether, polyethylene glycol dimethyl ether, 1,2-diethoxyethane, diethylene glycol diethyl ether, triethylene glycol diethyl ether, tetraethylene glycol diethyl ether, polyethylene glycol diethyl ether, acetonitrile, dimethyl formamide and dimethyl sulfoxide or mixture(s) thereof; protic polar organic solvents such as alcohols, for example methanol, ethanol, n-propanol, isopropanol, n-butanol, sec-butanol, isobutanol and tert-butanol or mixture(s) thereof.

In another embodiment of this aspect, the step b) can be performed at temperature selected from 0° C. to 10° C.

In another embodiment of this aspect, compound of Formula X can be isolated from the reaction mixture by extracting it with ether solvent (e.g. diethyl ether).

Accordingly, the compound of Formula VII in its salt form is reacted with an active ester of acid of Formula VIII in presence of base and organic solvent at temperature selected from about 5° C. to about 40° C. The reaction is performed at pH range of 5 to 7. The so produced compound of Formula VI is then isolated from the reaction mixture.

The compound of Formula VI is treated with base in polar organic solvent and water to produce the desired methyl [(2S)-3,3-dimethyl-1-({(1S)-1-[(2R)-oxiran-2-yl]-2-phenylethyl}amino)-1-oxobutan-2-yl]carbamate represented by Formula X.

The so produced compound of Formula X is then isolated from the reaction mixture after neutralizing the reaction mixture by adding sodium dihydrogen orthophosphate solution into it. Ether (e.g. diethyl ether) can be added to the reaction mixture to extract the compound of Formula X from the organic layer. The extracted compound of Formula X can be purified using hydrocarbon solvent (e.g. hexane).

The compound of Formula X can be further used as an intermediate in the preparation of Atazanavir or salt thereof.

A fifth aspect of the present invention provides threo-4-halo-3-hydroxy-2-amino-1-substituted butane derivative represented by general Formula II

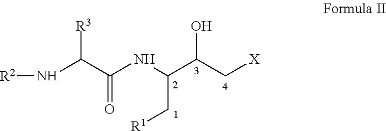

wherein

R¹ is phenyl,

R² is hydrogen or amino protecting groups,

R³ is secondary or tertiary lower alkyl and

X is chlorine, bromine, fluorine or iodine.

In an embodiment of this aspect, the compound of Formula II can be in (2S,3R) or (2R,3S) configuration.

In another embodiment of this aspect, the carbon atom bonded to the radical R³ in Formula II can be in (R)-, (S)- or (R,S)-configuration.

Another embodiment of this aspect provides methyl [(2S)-1-{[(2S,3R)-4-chloro-3-hydroxy-1-phenylbutan-2-yl]amino}-3,3-dimethyl-1-oxobutan-2-yl]carbamate represented by Formula VI

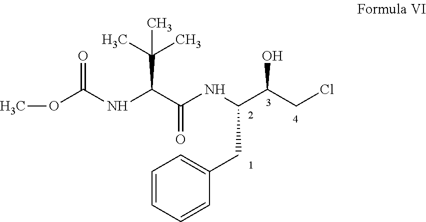

In another embodiment of this aspect, the compounds of Formula II and VI can be used as intermediates for Atazanavir or its salt preparation.

The preparation method of compounds of Formula II and VI are described hereinabove in first and third aspect, respectively, of the present invention.

While the present invention has been described in terms of its specific aspects, certain modifications and equivalents will be apparent to those skilled in the art and are intended to be included within the scope of the present invention.

In the following section aspects are described by way of examples to illustrate the processes of the invention. However, these are not intended in any way to limit the scope of the present invention. Several variants of these examples would be evident to persons ordinarily skilled in the art.

### Preparation of Starting Materials

### (2S)-2-[(methoxycarbonyl)amino]-3,3-dimethylbutanoic acid

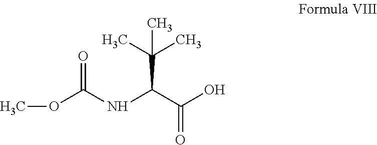

23.5 ml of methyl chloroformate was added over a period of 20 minutes to a solution of 20 g of 2(S)-amino-3,3-dimethyl-butyric acid in a mixture of 252 ml of 2N aqueous sodium hydroxide solution and 80 ml of dioxane and the reaction solution was heated at 60° C. for 14 hours. It was cooled to room temperature and then washed 2 times with methylene chloride. The aqueous phase was acidified to pH 2 with 4N aqueous hydrochloric acid and extracted three times with ethyl acetate. The organic extracts were combined, dried (Na₂SO₄) and concentrated by evaporation, the product started to solidify. The solidified solid was digested with hexane yielded (2S)-2-[(methoxycarbonyl)amino]-3,3-dimethylbutanoic acid in the form of a white powder.

M.p. 106°-108° C.

### Active Ester of (2S)-2-[(methoxycarbonyl)amino]-3,3-dimethylbutanoic acid

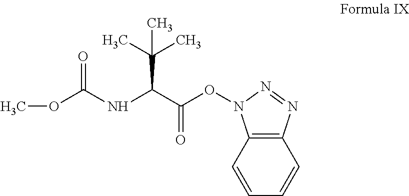

To a 3000 mL, 3-neck round bottom flask fitted with mechanical stirrer, addition funnel, nitrogen inlet, and temperature probe was added (2S)-2-[(methoxycarbonyl)amino]-3,3-dimethylbutanoic acid (VIII; 77.2 g), 1-hydroxybenzotriazole (HOBT) (60.8 g), and N-ethyl-N′-dimethylaminopropyl carbodiimide (EDC; 82.0 g) followed by CH₂Cl₂ (880 ml) and the mixture was stirred at ambient temperature (18-25° C.) until formation of the active ester was completed, as judged by HPLC.

### Example 1

### Preparation of methyl [(2S)-1-{[(2S,3R)-4-chloro-3-hydroxy-1-phenylbutan-2-yl]-amino}-3,3-dimethyl-1-oxobutan-2-yl]-carbamate (Formula VI)

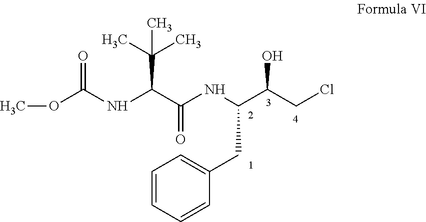

To (2S)-2-[(methoxycarbonyl)amino]-3,3-dimethylbutanoic acid (VIII; 92.19 g), HOBT (72.13 g), EDC hydrochloride (98.289 g) and dichloromethane (800 ml) were added at 20-25° C. and the solution was stirred for 3 hours at 20-25° C. A solution of K₂HPO₄ (120 g) in de-ionized water (800 ml) was added to the solution at 20-25° C. and then it was cooled to 10-15° C.

Another prepared solution of (2S,3R)-2-amino-4-chloro-1-phenylbutan-3-ol (Formula-VII) (100 g) in de-ionized water (400 ml) was drop-wise added to the cooled (10-15° C.) solution over a period of 1-2 hours. The reaction mixture so prepared was stirred at ambient temperature for 13-14 hours maintaining pH of the mixture in the range of 5 to 7. Completion of the reaction was monitored by TLC (Thin layer chromatography). After completion of the reaction, organic layer was separated and aqueous sodium hydroxide solution (40 g of NaOH dissolved in 800 ml of de-ionized water) was added to it at 10-15° C. The solution was then stirred for 30 minutes and again organic layer was separated. The organic layer was washed with 5% diluted hydrochloride solution (750 ml) at 10-15° C. and then with de-ionized water (800 ml). The solvent was completely removed from the organic layer under vacuum at not more then 45° C. to obtain the titled compound of Formula VI.

Yield (w/w)=1.45

### Example 2

### Preparation of methyl [(2S)-3,3-dimethyl-1-({(1S)-1-[(2R)-oxiran-2-yl]-2-phenylethyl}amino)-1-oxobutan-2-yl]carbamate (Formula X)

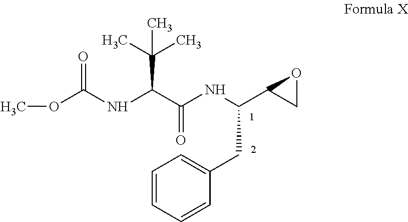

To methyl [(2S)-1-{[(2S,3R)-4-chloro-3-hydroxy-1-phenylbutan-2-yl]amino}-3,3-dimethyl-1-oxobutan-2-yl]carbamate (VI; 100 g), tetrahydrofuran (450 ml), ethanol (260 ml) and de-ionized water (87 ml) were added at ambient temperature. The solution was cooled to 0-5° C. and then aqueous KOH solution (39 g of KOH dissolved in 39 ml de-ionized water) was added to it at 0-5° C. The resultant reaction mixture was stirred at 0-5° C. for 2-3 hours. Completion of the reaction was monitored by TLC (Thin layer chromatography). After completion of the reaction, 6% sodium dihydrogen orthophosphate solution (725 ml) and diethyl ether (750 ml) were added to the reaction mixture at 0-5° C. and the solution was stirred for 10-15 minutes. The organic layer so formed was separated and de-ionized water (500 ml) was added to it at ambient temperature and stirred for 2 minutes. The organic layer was again separated and solvent was completely recovered under vacuum. To the residue, hexanes (300 ml) were added and it was stirred for 30 minutes at 5-10° C. The solid, so obtained, was washed with hexanes (100 ml) and dried at 35-40° C. under vacuum to obtain the titled compound of Formula X.

Yield (w/w)=0.803

We claim:

1. A process for the preparation of 4-halo-3-hydroxy-2-amino-1-substituted butane derivative represented by Formula II 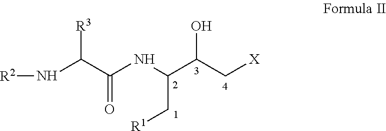
which comprises reacting compound of Formula III or salt thereof 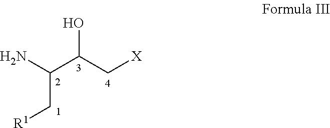
with an active ester of acid of Formula IV 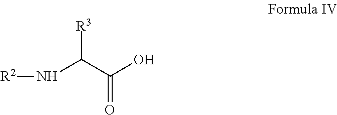
wherein
R¹ is phenyl,
R² is hydrogen or amino protecting groups,
R³ is secondary or tertiary lower alkyl and
X is chlorine, bromine, fluorine or iodine.

2. The process according to claim 1 wherein the reaction is performed in presence of base and organic solvent.

3. A process for preparation of threo-3,4-epoxy-2-amino-1-substituted butane derivative represented by Formula I: 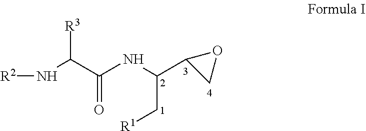
which comprises
a) reacting compound of Formula III or salt thereof 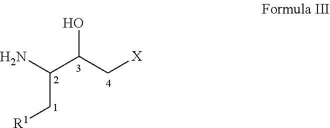
with active ester of acid of Formula IV 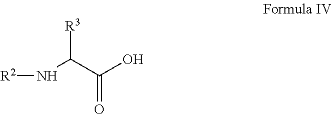
to produce 4-halo-3-hydroxy-2-amino-1-substituted butane derivative of Formula II 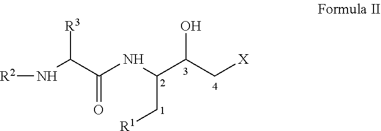
b) treating compound of Formula II produced in step a) with base
c) isolating threo-3,4-epoxy-2-amino-1-substituted butane derivative (I) from the reaction mixture of step b)
wherein
R¹ is phenyl,
R² is hydrogen or amino protecting groups,
R³ is secondary or tertiary lower alkyl and
X is chlorine, bromine, fluorine or iodine.

4. The process according to claim 3 wherein the step a) reaction is performed in presence of base and organic solvent.

5. The process according to claim 1 or 3 wherein active ester of acid of Formula IV is prepared by reacting the acid with coupling agent selected from the group comprising of O-(1,2-dihydro-2-oxo-1-pyridyl)-N,N,N¹,N¹-tetramethyluronium-tetrafluoro-borate (TPTU), 1-hydroxybenzotriazole (HOBT) and N-ethyl-N′-dimethylaminopropyl carbodiimide (EDC).

6. The process according to claim 1 or 3 wherein the active ester is represented by general Formula V

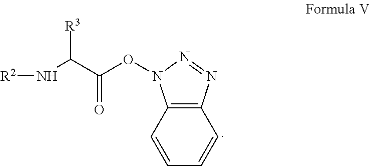

7. The process according to claim 3 wherein the step b) reaction is performed in presence of polar organic solvent optionally with water.

8. A process for the preparation of methyl [(2S)-1-{[(2S,3R)-4-chloro-3-hydroxy-1-phenylbutan-2-yl]amino}-3,3-dimethyl-1-oxobutan-2-yl]carbamate represented by Formula VI 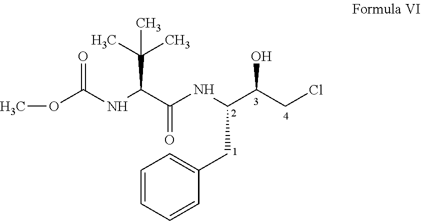
which comprises reacting (2S,3R)-2-amino-4-chloro-1-phenylbutan-3-ol represented by Formula VII or salt thereof 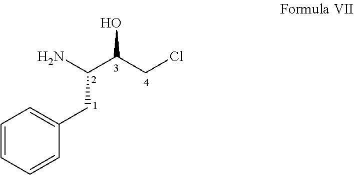
with an active ester of (2S)-2-[(methoxycarbonyl)amino]-3,3-dimethylbutanoic acid represented by Formula VIII.

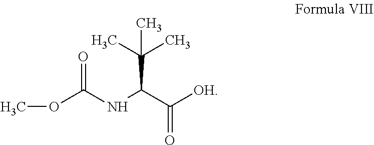

9. The process according to claim 8 wherein the reaction is performed in presence of base and organic solvent.

10. A process for the preparation of methyl [(2S)-3,3-dimethyl-1-({(1S)-1-[(2R)-oxiran-2-yl]-2-phenylethyl}amino)-1-oxobutan-2-yl]-carbamate represented by Formula X: 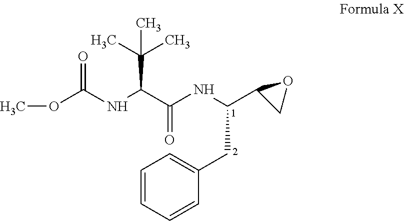
which comprises
a) reacting (2S,3R)-2-amino-4-chloro-1-phenylbutan-3-ol represented by 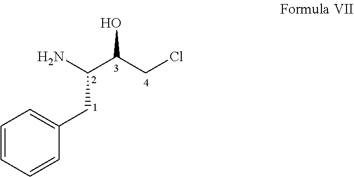
with an active ester of (2S)-2-[(methoxycarbonyl)amino]-3,3-dimethylbutanoic acid represented by Formula VIII 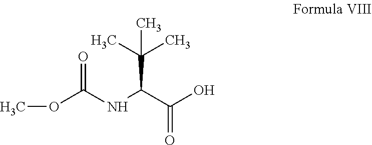
to produce methyl [(2S)-1-{[(2S,3R)-4-chloro-3-hydroxy-1-phenylbutan-2-yl]amino}-3,3-dimethyl-1-oxobutan-2-yl]-carbamate represented by Formula VI 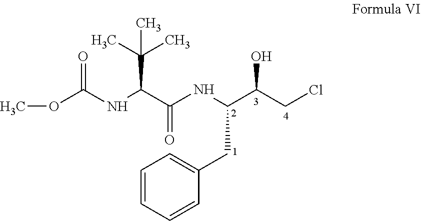
b) treating compound of Formula VI produced in step a) with base
c) isolating methyl [(2S)-3,3-dimethyl-1-({(1S)-1-[(2R)-oxiran-2-yl]-2-phenylethyl}amino)-1-oxobutan-2-yl]carbamate represented by Formula X from the reaction mixture of step b).

11. The process according to claim 10 wherein the step a) reaction is performed in presence of base and organic solvent.

12. The process according to claim 8 or 10 wherein active ester of acid of Formula VIII is prepared by reacting the acid with coupling agent selected from the group comprising of O-(1,2-dihydro-2-oxo-1-pyridyl)-N,N,N¹,N¹-tetramethyluronium-tetrafluoro-borate (TPTU), 1-hydroxybenzotriazole (HOBT) and N-ethyl-N′-dimethylaminopropyl carbodiimide (EDC).

13. The process according to claim 8 or 10 wherein the active ester is represented by Formula IX

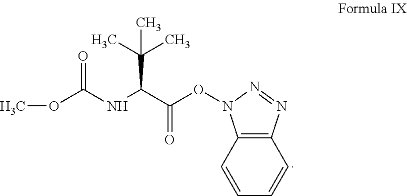

14. The process according to claim 10 wherein the step b) reaction is performed in presence of polar organic solvent optionally with water.

15. A threo-4-halo-3-hydroxy-2-amino-1-substituted butane compound represented by general Formula II 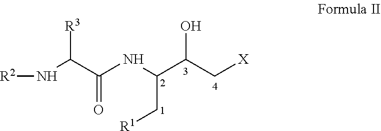
wherein
R¹ is phenyl,
R² is hydrogen or amino protecting groups,
R³ is secondary or tertiary lower alkyl and
X is chlorine, bromine, fluorine or iodine.

16. The compound according to claim 15 wherein the compound is methyl [(2S)-1-{[(2S,3R)-4-chloro-3-hydroxy-1-phenylbutan-2-yl]amino}-3,3-dimethyl-1-oxobutan-2-yl]carbamate represented by Formula VI

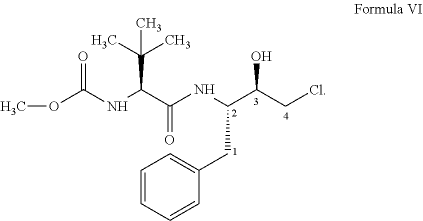

17. Use of compound of Formula II or VI for the preparation of atazanavir or salt thereof.

18. Use of compound of Formula I or X for the preparation of atazanavir or salt thereof.
